(19) United States
(12) Patent Application Publication
(10) Pub. No.: US 2007/0269509 A1
(43) Pub. Date: Nov. 22, 2007

(54) Enteric Coated Pharmaceutical Oral Formulations Comprising Acid-Labile Active Substances, and a Method Thereof

(76) Inventors: Woo Heon Song, Gyeonggi-do (KR); Hun Sik Wang, Gyeonggi-do (KR); Min Chang Kwon, Seoul (KR); Jun Sang Park, Gyeonggi-do (KR)

Correspondence Address: SUGHRUE MION, PLLC, 2100 PENNSYLVANIA AVENUE, N.W., SUITE 800, WASHINGTON, DC, 20037, US

(21) Appl. No.: 10/596,187

(22) PCT Filed: Feb. 8, 2006
(86) PCT No.: PCT/KR06/00459

§ 371 Date: May 21, 2007

(30) Foreign Application Priority Data
Feb. 14, 2005 (KR) 10-2005-0012104

(51) Int. Cl. A61K 9/30; A61K 31/4436; A61K 9/20

(52) U.S. Cl. 424/465; 424/475; 514/338

(57) ABSTRACT

This invention relates to an oral pharmaceutical formulation formed by a direct coating of an enteric layer containing polyethylene glycol as a plasticizer on a core containing an acid-labile pantoprazole, and its manufacturing method. The enteric-coated oral pharmaceutical formulation of this invention, which combines directly a core containing an acid-labile pantoprazole and an enteric layer in the absence of an inert intermediate layer, is able to improve the storage stability of the acid-labile pantoprazole and maximize the bioavailability and oral absorption rates via preventing related substances from increasing.

### TECHNICAL FIELD

This invention relates to an enteric-coated oral pharmaceutical formulation containing an acid-labile pantoprazole to prevent the declining pharmacologic action of pantoprazole and the occurrence of related substances.

### BACKGROUND ART

One class of acid-labile active ingredients is the group of pharmaceutical active ingredients such as benzimidazole derivatives, called “proton pump inhibitors”. These compounds are known to be effective for prevention and treatment of gastric-acid related diseases, including e.g., gastric ulcers and duodenal ulcers, reflex esophagitis, and infections associated with Helicobacter pylori. Among benzimidazole compounds, pantoprazole is extremely unstable in the acidic condition.

In order to avoid contact between the acid-labile compound and the acidic gastric fluid following oral administration, a pharmaceutical oral formulation having an enteric layer on a core has been developed.

The enteric coating of the oral pharmaceutical formulation, however, presents its own problems as enteric polymers have acidic moiety, which can cause the decomposition of the acid-labile compound during preparing and storage of formulation, thus leading to the reduced pharmacologic action.

In order to avoid such problem, an inert intermediate layer, which is not acidic, is often required between the core and the enteric layer.

FIG. 1 shows the conventional oral pharmaceutical formulation having a core containing acid-labile pharmaceutical compound (101), inert intermediate layer (102) and enteric layer (104). FIG. 2 shows another example of the conventional oral pharmaceutical formulation having two inert intermediate layers (202, 203).

Many alternative dosage forms have been proposed in the literatures.

The Korean Patent Registration No. 88473 describes a process for manufacturing an oral pharmaceutical preparation of a core formulation comprising an acid-labile drug and an alkaline substance, wherein the process consists of (a) preparing the core (101) by mixing the acid-labile drug with the alkaline substance; coating the core with an inert intermediate layer (102) containing water-soluble polymers; and coating the outer surface (103) with an enteric coating agent.

The Korean Patent Registration No. 254021 describes an oral pharmaceutical composition comprising (a) a core (101) containing an acid-labile pantoprazole, polyvinylpyrrolidone and/or hydroxypropylmethyl cellulose as binder, and mannitol or a basic inorganic compound as a filler, if necessary, (b) an inert water-soluble intermediate layer (102) that surrounds the core, and a gastric juice-resistant enteric layer (104).

The Korean Patent Registration No. 43430 describes an oral pharmaceutical preparation comprising a nucleus (101) containing an acid-labile drug; a first layer (102) coating a nuclear with mixture of a poorly water-soluble coating material such as ethyl cellulose or polyvinyl acetate and a poorly water-soluble microgranules selected from the group consisting of magnesium oxide, silicon dioxide, calcium silicate, magnesium hydroxide, magnesium carbonate, aluminum hydroxide, calcium stearate, magnesium stearate and sucrose fatty acid ester; a second enteric layer (104) coating the first layer with a enteric coating material.

A literature (Chem. Pharm. Bull. 51(9) 1029-1035(2003)) describes an oral pharmaceutical preparation of microgranules comprising an active layer (101) containing an acid-labile lansoprazole and magnesium carbonate, an intermediate layer (102) containing hydroxypropylmethyl cellulose, and an enteric layer (104) containing Macrogol 6000.

The Korean Patent Application No. 1991-18270 describes an oral pharmaceutical preparation comprising a core (201) containing an acid-labile drug, more than two intermediate layers including water-soluble inert layer (202) and inert layer containing a water-soluble polymer and poorly water-soluble alkaline microgranules (203), and enteric layer (204).

The Korean Patent Application No. 1990-12623 describes an oral pharmaceutical preparation comprising a core (201), a water-soluble coating layer (202), a water absorption layer (203), and enteric coating layer (204).

The essential requirement of an inert intermediate layer in an oral pharmaceutical formulation to enhance the storage stability may increase the complexity and the cost of manufacture process of the formulation involving acid-labile compounds.

As illustrated in FIG. 3, therefore, intensive studies have been made to directly coat an enteric layer (304) on a core (301) containing an acid-labile drug. These approaches are intended for improving the stability of an oral preparation during storage via an appropriate formulation of a core.

The U.S. Pat. No. 6,602,522 describes a pharmaceutical composition comprising a tableted core (301) having an uncoated granulation of an acid-labile drug, a pharmaceutically acceptable alkaline agent, at least one water-soluble binder and at least one water-insoluble binder, wherein a single coating (304) comprising an enteric coating agent is provided around the tableted core.

The Korean Patent Application No. 1999-7012021 describes an oral pharmaceutical preparation of a core formulation (301) having an acid-labile drug and a mixture of compounds selected from the group consisting of crospovidone, sodium hydroxide, potassium hydroxide, and sodium carbonate, wherein a single coating (304) comprising an enteric coating agent is provided around the tableted core.

Besides, the Korean Patent Registration No. 314351 describes an oral pharmaceutical composition having a core (301) comprising benzimidazole derivative resin salts formed by ionization between benzimidazole derivatives and an anionic exchange resin selected from the group consisting of cholestyramine resin and DOWEX resin, wherein the surface of the core is coated with an enteric coating agent (304) having an acidic group substitution rate of less than 30%.

WO 2000/78284 is a published PCT application describing a pharmaceutical composition having a core (301) comprising an acid-labile benzimidazole derivative, wherein a single coating comprising an enteric coating agent (304) at more than pH 6.5 is provided around the tableted core.

However, problems still exist in that the decomposition of active ingredients by an enteric coating agent cannot be effectively prevented, although the most stable core is formulated using an acid-labile drug and the prior arts. Furthermore, the fact that the increase of related substances produced by the decomposition of active compound associated with an enteric coating agent irrespective of the presence of an inert layer causes a concern for the storage stability.

The related substance, which is reported to generate during the manufacturing process and storage, has been recognized as one of the most important factors to determine the quality of a pharmaceutical formulation. As the related substance tends to induce an unwanted toxicity and pharmacologic activity, a pharmaceutical manufacturer who intends to obtain a product license is required to submit some data related to not only chemical indentification but also pharmacologic and toxicity data of related substances. According to the ICH guideline, a pharmaceutical manufacturer should submit safety data if the content of individual related substance exceed 0.5%.

Thus, a stricter regulation on the specification of related substances in preparation of an oral pharmaceutical formulation containing an acid-labile drug has been applied. In the case of pantoprazole (Pantoloc tablet, Pacific Pharm, Korea), benzimidazole-derived proton pump inhibitor, the amount of individual related substance should be not more than 0.5% and the sum of related substances should be not more than 1.0%.

In the other hand, a plasticizer is added to polymeric solution for the coating process of pharmaceutical dosage forms in order to prepare a coating layer efficiently. A plasticizer reduces the glass transition temperature of polymers resulting in more tight coalescence of polymers at the surface of a pharmaceutical dosage form.

In case of the enteric coating, the addition of a plasticizer has been applied generally to accomplish the same purpose. The primary aim of formulating an enteric coating layer is that acid-labile active ingredients may pass through the acidic stomach unharmed and then be released in the intestinal tract where they may be absorbed into the general blood circulation. To this end, extremely hydrophobic plasticizers such as triethylcitrate (TEC), dibutyl sebacate (DBS) and diethylphthalate (DEP) have been added to the enteric coating agent to inhibit the penetration of gastric juices, instead of hydrophilic plasticizers such as polyethylene glycol (PEG) and propylene glycol (PG). In particular, triethylcitrate is known as the most preferred plasticizer for mathacrylic acid copolymer (Eudragit® L30D or L30D-55), and the use of other plasticizers may be problematic [Product brochure (Eudragit®, Degussa)]. Nevertheless, such hydrophobic plasticizers have failed to completely inhibit the decomposition of active ingredients and generation of related substances associated with the enteric coating agent during storage.

In this context the inventors discovered that the formation of an enteric layer containing polyethylene glycol as a plasticizer on a core comprising an acid-labile pantoprazole without an inert intermediate layer might not only improve the storage stability through inhibiting the decomposition of an acid-labile pantoprazole, but also contribute to significant reduction of related substances. As a consequence the inventors consummated this invention.

### DISCLOSURE

[Technical Problem]

An object of this invention is to provide an enteric-coated oral pharmaceutical formulation to improve the storage stability of the acid-labile pantoprazole and maximizing the bioavailability and oral absorption rates via preventing related substances from increasing, although an enteric layer is directly coated on a core containing an acid-labile pantoprazole in the absence of an inert intermediate layer.

[Technical Solution]

An object of this invention is to provide an enteric-coated oral formulation formed by a direct enteric coating on a core tablet containing an acid-labile pantoprazole by using polyethylene glycol as a plasticizer, and its manufacturing method.

This invention is described in more detail as set forth hereunder.

This invention relates to an enteric-coated oral pharmaceutical formulation, wherein a core containing an acid-labile pantoprazole is coated with an enteric coating composition containing polyethylene glycol as a plasticizer.

The core of this invention may contain an acid-labile pantoprazole, a mixture of pharmaceutical acceptable binder, diluent, disintegrant and lubricant and an alkaline reacting compound.

The examples of the acid-labile drugs, which are decomposed in the acidic condition, include substituted benzimidazole derivatives such as rabeprazole, omeprazole, pantoprazole, and lansoprazole. This invention relates to an enteric-coated oral pharmaceutical formulation containing an acid-labile pantoprazole. According to this invention, pantoprazole or its alkaline salts (e.g., sodium, potassium, magnesium, or calcium) may be employed to prepare an enteric-coated oral formulation. Further, the oral pharmaceutical formulation of this invention may contain a therapeutically effective amount per tablet in the range of 5-35 w/w % based on the total weight of a core.

A binder for use in the core of this invention may include a low-viscosity hydroxypropylmethyl cellulose. The preferred substitution profile of hydroxypropylmethyl cellulose is that the substitution rates of methoxy and hydroxypropoxy groups are in the range of 28-30% and in 7-12%, respectively. The preferred viscosity is in the range of 3-15 cp, more preferably in 6 cp. The quantity of the binder comprises 10-20 w/w % based on the total weight of a core.

A diluent for use in the core of this invention may include lactose, mannitol or mixture thereof. The quantity of the diluent comprises 10-40 w/w % based on the total weight of a core.

A disintegrant for use in the core of this invention may include crospovidone, low-substituted hydroxypropyl cellulose, sodium lauryl sulfate or mixture thereof. The quantity of the disintegrant comprises 2-40 w/w % based on the total weight of a core.

A lubricant for use in the core of this invention may include stearic acid or its salts, talc, magnesium silicate, glyceryl behenate, sodium stearyl fumarate or mixture thereof. The quantity of the lubricant comprises 0.5-5 w/w % based on the total weight of a core.

An alkaline compound may include alkali metal salts such as phosphate, silicate, carbonate, hydroxide, ammonium salt, basic amino acids or mixture thereof. The quantity of the alkaline compound comprises 5-15 w/w % based on the total weight of a core.

The enteric layer of this invention contains essentially enteric polymers and polyethylene glycol as a plasticizer. Polyethylene glycol having an average molecular weight of 400-8000 may be employed, and it is preferred to employ polyethylene glycol having an average molecular weight of 6000.

The quantity of polyethylene glycol comprises 5-20 w/w % based on the total weight of enteric polymers, and it is preferred to employ polyethylene glycol in the range of 10-15 w/w % based on the total weight of enteric polymers. Further, the enteric layer may optionally contain an alkaline compound and pharmaceutically acceptable additives. The quantity of the enteric layer comprises 15-40 w/w % based on the total weight of a core.

In general, the addition of a plasticizer to an enteric layer is a conventional formulation technology. The primary aim of formulating an enteric layer is to ensure that acid-labile drugs may pass through the acidic stomach unharmed and then be released in the intestinal tract where they may be absorbed into the general blood circulation. Thus, extremely hydrophobic plasticizers have been commonly employed for formulating the enteric layer.

Notwithstanding this, this invention does not introduce hydrophobic plasticizers such as triethylcitrate (TEC), dibutyl sebacate (DBS) or diethylphthalate (DEP) for formulating the enteric layer, since they may aggravate the storage stability of an acid-labile compound.

This invention is characterized by providing an oral pharmaceutical formulation, wherein polyethylene glycol is essentially employed for formulating the enteric layer that is directly coated on a core containing an acid-labile drug to achieve the purpose of blocking the adverse impact of the enteric coating material on an acid-labile drug.

The amount of the related substance, which is reported to generate during the manufacturing process and storage of a pharmaceutical ingredient and a pharmaceutical product, increases remarkably in preparing a pharmaceutical formulation containing acid-labile drug resulting in an unwanted toxicity and pharmacologic activity,

According to this invention, the use of polyethylene glycol as a plasticizer for formulating the enteric layer may significantly reduce the generation of related substances.

The enteric polymers of this invention may include methacrylic acid copolymer (Eudragit® L30D or L30D-55), hydroxypropyl methylcellulose acetate succinate, polyvinyl acetate phthalate or other enteric polymers that may be suspended in water. Preferably, methacrylic acid copolymer may be employed. The quantity of the enteric polymer comprises 40-80 w/w % based on the total weight of an enteric coating layer.

The enteric coating layer of this invention may optionally contain an alkaline compound. The alkaline compound of this invention may include alkali metal salts such as phosphate, silicate, carbonate, hydroxide, ammounium salt or the mixture thereof. The alkaline compound may be added to the solution or suspension for the enteric layer in an amount to adjust pH to 5.0-6.0, preferably to 5.5.

The pharmaceutically acceptable additives include talc, sodium lauryl sulfate, titanium oxide or iron oxide. In addition, water may be employed as a solvent.

According to this invention, specific color layer may be separately provided to the enteric layer that is understood by person having ordinary skill in the art.

In accordance with another aspect, this invention provides a process for manufacturing an oral pharmaceutical formulation containing polyethylene glycol as a plasticizer in the enteric layer.

The enteric-coated oral pharmaceutical formulation of this invention may be prepared by the following steps of including a) mixing the acid-labile drug with the commonly used excipients in the pharmaceutical field such as a binder, a diluent, a disintegrant, a lubricant and/or an alkaline compound to prepare a variety of cores (e.g., tablet, granule, microgranule, or capsule) in a common manner; b) dissolving or suspending an enteric polymer and polyethylene glycol as a plasticizer in a solvent to prepare an enteric suspension, and c) spraying the enteric suspension to the core to form an enteric layer.

[Advantageous Effects]

The enteric coated oral pharmaceutical formulation of this invention is prepared by directly coating an enteric layer containing polyethylene glycol as a plasticizer on a core containing an acid-labile pantoprazole.

The direct coating of the enteric layer on the core does not cause the decomposition of pantoprazole, thus ensuring better storage stability for a long-term period as well as a significant reduction of related substances. The oral pharmaceutical formulation of this invention may decrease the complexity and the cost of the manufacturing process involving acid-labile pantoprazole.

### DESCRIPTION OF DRAWINGS

FIG. 1 shows an enteric-coated oral pharmaceutical formulation comprising (a) a core (101) containing an acid-labile drug, an inert intermediate layer (102) and an enteric layer (104).

FIG. 2 shows an enteric-coated oral pharmaceutical formulation comprising (a) a core (201) containing an acid-labile drug, an inert intermediate layer I (202), an inert intermediate layer II (203) and an enteric coating layer (204).

FIG. 3 shows an enteric-coated oral pharmaceutical formulation prepared by directly coating an enteric layer (304) on a core (301) containing an acid-labile drug.

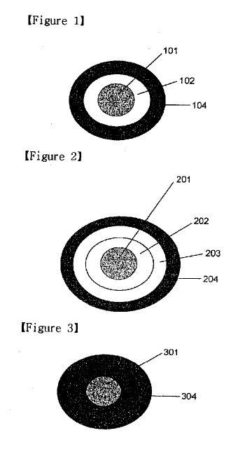

### BEST MODE

This invention will now be described by reference to the following examples and experimental examples which are merely illustrative and which are not to be construed as a limitation of the scope of this invention.

### EXAMPLE 1

### Preparation of Enteric-Coated Tablets Containing Polyethylene Glycol as a Plasticizer

An enteric-coated tablet containing polyethylene glycol as a plasticizer was prepared based on the formula, as described in the following Tables 1 and 2.

Pantoprazole sodium sesquihydrate (270.6 g), dried sodium carbonate (120.0 g), hydroxypropylmethyl cellulose 2910 (88.0 g) and crospovidone (252.0 g) were mixed in a vertical granulator (Korea machinery Co.), granulated by addition of the binding solution in which hydroxypropylmethyl cellulose 2910 (44.0 g) dissolved in purified water (300.0 g), and passed through #14 sieve. The granule was dried at 60° C. and passed through #18 sieve. Crospovidone (48.0 g), lactose (129.6 g), talc (10.2 g) and sodium stearyl fumarate (30.0 g) were added to the granule and mixed. The final mixture was compressed into tablets using a rotary tabletting machine (Erweka Co.).

An enteric coating suspension was prepared in the following manner:

Methacrylic acid-acrylic acid ethyl copolymer suspension by 30 wt. % (Eudragit® L30D55) (1146.0 g) was mixed with purified water (573.0 g), and stirred.

Polyethylene glycol 6000 (34.5 g), sodium hydrogen carbonate (10.2 g) and sodium lauryl sulfate (3.0 g) were dissolved into the purified water (1025.0 g), and this solution added to polymeric dispersion. Then, a homogeneous suspension of titanium oxide (45.0 g) and talc (163.5 g) were added with stirring, and passed through #100 sieve.

Previously prepared core tablets were put into a coating machine (Sejong Machinery Co.). The enteric coating suspension was sprayed onto the core tablets in an amount of about 25 wt. % based on the total weight of core tablet.

TABLE 1
Component ratio of core tablets

| Component | Weight per tab. (mg) | Wt. % |
|---|---|---|
| Pantoprazole sodium sesquihydrate | 45.10 | 27.3 |
| Hydroxypropylmethyl cellulose 2910 | 22.00 | 13.3 |
| Dried sodium carbonate | 20.00 | 12.1 |
| Crospovidone | 50.00 | 30.2 |
| Lactose | 21.60 | 13.1 |
| Talc | 1.70 | 1.0 |
| Sodium stearyl fumarate | 5.00 | 3.0 |
| Purified water | 50.00 | |
| Total | 165.40 | 100.0 |

TABLE 2
Component ratio of enteric coating suspension

| Component | Wt. % |
|---|---|
| Methacrylic acid-acrylic acid ethyl copolymer | 11.46 |
| Polyethylene glycol 6000 | 1.15 |
| Talc | 5.45 |
| Titanium oxide | 1.50 |
| Sodium hydrogen carbonate | 0.34 |
| Sodium lauryl sulfate | 0.10 |
| Purified water | 80.00 |
| Total | 100.0 |

### COMPARATIVE EXAMPLE 1

### Preparation of Enteric-Coated Tablets Containing Triethylcitrate as a Plasticizer

An enteric-coated tablet containing triethylcitrate as a plasticizer was prepared based on the formula, as described in the following Tables 3 and 4.

Pantoprazole sodium sesquihydrate (270.6 g), dried sodium carbonate (120.0 g) and crospovidone (252.0 g) were mixed in a vertical granulator, granulated by addition of the binding solution in which hydroxypropylmethyl cellulose 2910 (48.0 g) dissolved in purified water (300.0 g), and passed through #14 sieve. The granule was dried at 60° C. and passed through #18 sieve. Crospovidone (48.0 g), lactose (129.6 g), talc (10.2 g) and sodium stearyl fumarate (30.0 g) were added to the granule and mixed. The final mixture was compressed into tablets using a rotary tabletting machine (Erweka Co.).

An enteric coating suspension was prepared in the following manner:

Methacrylic acid-acrylic acid ethyl copolymer suspension by 30 wt. % (Eudragit® L30D55) (1429.0 g) was mixed with purified water (715.0 g), and stirred. Separately, a homogeneous suspension of triethylcitrate (42.9 g) and talc (128.4 g) in purified water (685.0 g) were added to the polymeric dispersion with stirring, and passed through #100 sieve.

Previously prepared core tablets were put into a coating machine (Sejong Machinery Co.). The enteric coating suspension was sprayed onto the core tablets in an amount of about 15 wt. % based on the total weight of a core tablet.

TABLE 3
Component ratio of tableted core

| Component | Weight per tab. (mg) | Wt. % |
|---|---|---|
| Pantoprazole sodium sesquihydrate | 45.10 | 29.9 |
| Hydroxypropylmethyl cellulose 2910 | 8.00 | 5.3 |
| Dried sodium carbonate | 20.00 | 13.3 |
| Crospovidone | 50.00 | 33.2 |
| Lactose | 21.60 | 14.3 |
| Talc | 1.50 | 1.0 |
| Sodium stearyl fumarate | 4.50 | 3.0 |
| Purified water | 50.00 | |
| Total | 150.70 | 100.0 |

TABLE 4
Component ratio of enteric coating suspension

| Component | Wt. % |
|---|---|
| Methacrylic acid-acrylic acid ethyl copolymer | 14.29 |
| Triethylcitrate | 1.43 |
| Talc | 4.28 |
| Purified water | 80.00 |
| Total | 100.0 |

### EXPERIMENTAL EXAMPLE 1

### Stability Test Under Stressed Condition

The enteric-coated tablets, so prepared from Example 1 and Comparative example 1, were stored at 60° C. for 1 month and analyzed by a high performance liquid chromatography to detect the changes in the contents of an original drug and related substances with the passage of time, under the conditions specified in Table 5.

TABLE 5

| | | Related |
|---|---|---|
| Category | Original substance | substance |
| Column | Zorbax Eclipse XDB-C18 | Zorbax Eclipse |
| | (4.6150 mm, 5 m) | XDB-C18 |
| | | (4.6150 mm, 5 m) |
| Mobile phase | pH 7.0 phosphate buffering | pH 7.0 phosphate |
| | solution:acetonitrile = | buffering solution: |
| | 65:35 | acetonitrile = 75:25 |
| Flow rate | 1.0 mL/min | 1.0 mL/min |
| Detected wavelength | UV 290 nm | UV 290 nm |
| Input | 10 L | 20 L |
| Total analysis time | 7 min | 50 min |

As shown in Table 6, the enteric-coated formulation containing triethylcitrate (Comparative example 1) indicated in the stressed condition that the contents of acid-labile pantoprazole sodium were decreased by about 11%, whereas those of the enteric-coated formulation containing polyethylene glycol (Example 1) were more than 97%. The direct coating of the enteric layer of this invention on the core in the absence of an inert intermediate layer contributed much to better storage stability of the acid-labile drug for a long-term period, when compared to Comparative example.

The incidence of related substances in Comparative example was about 9%, whereas that in Example 1 was about 1%. In this context, the enteric-coated formulation of this invention has proven to have an excellent stability profile.

TABLE 6

| | Baseline (contents, %) | | Day 7 (contents, %) | | Day 14 (contents, %) | | Month 1 (contents, %) | |
|---|---|---|---|---|---|---|---|---|
| | | Related | | Related | | Related | | Related |
| | Original | substance | Original | substance | Original | substance | Original | substance |
| | drug (%) | (%) | drug (%) | (%) | drug (%) | (%) | drug (%) | (%) |
| Example 1 | 100.50 | 0.23 | 100.10 | 0.46 | 99.60 | 0.63 | 97.7 | 1.12 |
| Comparative | 100.77 | 0.31 | 97.10 | 1.81 | 93.32 | 4.35 | 86.19 | 8.52 |
| example 1 | | | | | | | | |

1. An oral pharmaceutical formulation comprising pantoprazole formed by a direct enteric coating layer comprising an enteric polymer and polyethylene glycol as an essential plasticizer, on a core containing pantoprazole or its alkaline metal salts as an active ingredient.

2. The oral pharmaceutical formulation comprising pantoprazole according to claim 1, wherein its core contains a low-viscosity hydroxypropylmethyl cellulose as a binder, lactose as a diluent, and crospovidone as a disintegrant.

3. The oral pharmaceutical formulation comprising pantoprazole according to claim 1, wherein the enteric polymer is methacrylic acid copolymer.

4. A method for manufacturing the oral pharmaceutical formulation comprising pantoprazole prepared by a process which includes forming a core comprising pantoprazole or its alkaline metal salts;
dissolving or suspending an enteric polymer and polyethylene glycol as an essential plasticizer in a solvent to prepare an enteric suspension;
and spraying the enteric suspension to the core to form an enteric coating layer, wherein another intermediate layer is not formed between the core and enteric coating layer.

5. The method for manufacturing the oral pharmaceutical formulation comprising pantoprazole according to claim 4, wherein its core contains a low-viscosity hydroxypropylmethyl cellulose as a binder, lactose as a diluent, and crospovidone as a disintegrant.

6. The method for manufacturing the oral pharmaceutical formulation comprising pantoprazole according to claim 4, wherein the enteric polymer is methacrylic acid copolymer.
